(19) United States
(12) Patent Application Publication
(10) Pub. No.: US 2017/0368378 A1
(43) Pub. Date: Dec. 28, 2017

(54) DRESSING FORMULATIONS TO PREVENT AND REDUCE SCARRING

(71) Applicant: MOKO THERAPEUTICS LLC, Vista, CA (US)

(72) Inventor: Brian C. KELLER, Antioch, CA (US)

(73) Assignee: MOKO THERAPEUTICS LLC, Vista, CA (US)

(21) Appl. No.: 15/701,207

(22) Filed: Sep. 11, 2017

Related U.S. Application Data
(63) Continuation of application No. 12/555,749, filed on Sep. 8, 2009, now abandoned.
Application No. 12/555,749 is a continuation-in-part of application No. PCT/US2008/056443, filed on Mar. 10, 2008.
(60) Provisional application No. 60/905,984, filed on Mar. 8, 2007.

(51) Int. Cl. A61Q 19/00; A61K 8/895; A61K 8/58; A61L 15/26; A61L 15/44; A61L 26/00

(52) U.S. Cl. CPC A61Q 19/00; A61L 15/44; A61L 26/0066; A61L 26/0019; A61K 8/585; A61L 15/26; A61K 8/895; A61L 2300/626

(57) ABSTRACT

Provided herein is a scar dressing formulation comprising a blend of a high molecular weight silicone elastomer crosspolymer and a silicone oil, wherein said silicone elastomer crosspolymer is in a volatile fluid. The formulation has a soft, silky feel without being greasy and dries quickly to form a durable, flexible scar dressing.

### RELATED APPLICATION

This application is a continuation-in-part of PCT Application No. PCT/US2008/056443, filed Mar. 10, 2008, and published in English on Sep. 12, 2008, which claims the benefit of priority of U.S. Provisional No. 60/905,984, now expired. Both applications are hereby incorporated by reference in their entirety.

### TECHNICAL FIELD

The present invention relates to scar dressings. More particularly, the present invention relates to compositions for dressing that prevent or reduce scarring comprising anhydrous silicone preparations applied directly to the scar.

### BACKGROUND ART

Scaring results from a normal physiological healing response after skin injury or incision. The skin wound healing process consists of three phases-inflammation, granulation and matrix remodeling. During the first phase, an inflammatory response is mounted, producing a cascade of biochemical reactions that result in vasodilation, exudate filling of the wound, and swelling at the site of injury. Neutrophil migration into the area of injury triggers phospholipase A2 (PLA2) release and prostaglandins production causing cellular and tissue damage. In the second phase, granulation takes place as macrophages secrete cytokines to promote granulated tissue formation. This new tissue consists of new epithelial tissue complete with new vasculature and blood supply. In phase three, matrix remodeling occurs as fibroblasts proliferate and manufacture collagen, elastin and other tissue building blocks in and around the wound site.

Hypertrophic scars represent a frequent but exaggerated response to healing. Clinically, hypertrophic scars are raised, red and often nodular. They occur in all skin areas but are most common in areas of thick skin. Frequently, hypertrophic scars develop within weeks of a burn, wound closure, wound infection, hypoxia and other traumatic skin injury. Collagen found in this type of scars in highly disorganized and forms whorl like arrangements rather than normal parallel orientation, that causes induration and elevation above the normal skin surface.

People with abnormal skin scarring face physical and psychological consequences that are frequently associated with substantial emotional and financial costs. Therefore, treatment and prevention of scarring remains a critical unmet need. Current treatment options range from no treatment at all (i.e., leaving the scar alone); invasive procedures and surgery such as intralesional corticosteroids, laser therapy and cryosurgery; and noninvasive management, particularly through topical medications. See, e.g., Zurada et al., J. Am. Acad. Dermatol. 55:1024-31 (2006). Most scar sufferers who elect to undergo treatment prefer to use noninvasive techniques because overall compliance is higher, the process is self-controlled and less painful.

One of the important materials used in noninvasive management of scars has been polydimethylsiloxone polymer gel sheeting or silicone gel sheeting. Since being introduced in 1982, topical silicone gel sheeting has been used to minimize the size, induration, erythema, pruritus, and extensibility of pre-existing hypertrophic scars with mixed results. See, e.g., Fette, Plastic Surg. Nurs. 26:87-92 (2006); de Oliveria et al., Dermatol. Surg. 27:721-26 (2001); Ricketts et al., Dermatol. Surg. 22:955-59 (1996). However, controlled studies have demonstrated no significant differences between gel wound dressing and silicone-based wound dressings. Moreover, the main drawback to silicone gel sheeting is a difficulty of use and thus, a high non-compliance. By their nature silicone gels are difficult to handle. They are soft and frangible and the gel sheets are thus easily torn in use. It has been proposed to improve the strength and ease of handling of silicone gel sheets by embedding therein during manufacture a support material such as a net of polyester or other fibers. Although this technique has resulted in an improvement in the ability to handle and apply the gel sheet it has been found that the sheet still has a tendency to fragment during application and in use. The sheeting also must be worn up to 24 hours a day for 2-4 months.

Seeking to avoid some of the constraints with silicone gel sheeting, liquid silicone gel products have also been tried. Liquid dimethicone products, for example, have the advantage of easy of use but again compliance is low due to the unappealing greasy, messy nature of liquid dimethicone. Attempts to reduce or eliminate the messy nature of silicone largely depend on complicated wound dressing formulations that lack the necessary conformability and long term flexibility necessary for most wounds.

Therefore, there remains a need for improved wound or scar dressing that is easily applied without inducing pain or further comprising the wound but results in a conforming and flexible dressing that provides sufficient protection to permit appropriate tissue regeneration at the site of injury.

### DISCLOSURE OF THE INVENTION

The present invention relates to scar dressings comprising silicone elastomer crosspolymers that are easily applied to closed wounds and rapidly cure at room temperature. The silicone crosspolymer mixture can readily be applied directly to closed wounds and when cured, provides a dressing that is easily applied to recently closed wounds, conformable and flexible. The dressings provided herein minimize further scarring, reduce potential infections, and diminish the intensity and duration of scar discoloration.

More particularly, provided herein is a scar dressing comprising a blend of a high molecular weight silicone elastomer crosspolymer and a silicone oil, wherein said silicone elastomer crosspolymer is in a volatile fluid. The silicone elastomer crosspolymer can be dimethicone-based or a blend of cylclohexasiloxane and cyclopentasiloxane. The silicone oil can be dimethicone, cyclomethicone or a mixture thereof.

The dressing has a soft, silky feel on the skin upon application and can be applied without producing further injury or discomfort. After the blend is applied to the closed wound, evaporation of the volatile diluent results in a “curing” of the silicone mixture to form a highly flexible dressing that can cover the closed wound or scar for extended periods of time. It can be occlusive. The formulation can be a scar treatment product that is easy to apply to tender scars, soothing to the scar tissue, painless, free from side effects and easy for the user to comply with over multiple applications because it is not greasy, goes on dry and occludes the scar for the best chance of continued healing without induration, hypertrophy and permanent disfiguration.

The dressings of the presently disclosed invention are useful as a delivery vehicle for a wide variety of compositions. The disclosed invention provides a non-greasy dressing for the topical application of non-aqueous formulations that will increase patient compliance and improve treatment outcomes. Compositions previously had to be formulated in greasy ointments, for example those that use petroleum-derived hydrocarbons, for proper and efficacious deliver can now be formulated in a pharmaceutically elegant easy to use, aesthetic dressing. This dressing is particularly useful as a drug vehicle because it has the right mixture of cross-polymer silicones and silicone oils that dissolve the drug substance and allow for better drug activity in the skin.

### MODES OF CARRYING OUT THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

Silicones are a group of completely synthetic polymers containing the recurring group —SiR₂O—, wherein R is a radical such as an alkyl, aryl, phenyl or vinyl group. The simpler silicones are oils of very low melting point, while at the other end of the scale of physical properties are highly crosslinked silicones which form rigid solids. Intermediate in physical properties between these two extremes are silicone elastomers such as gels and rubbers. When the silicones are formed by crosslinking a mixture of two or more silicones, the molecular weights of the various components and/or their degree of substitution by reactive groups may be different. This allows mixtures having different physical properties to be formed merely by varying the proportions of the components.

Provided herein is a scar (or wound) dressing comprising a blend of a high molecular weight silicone elastomer crosspolymer and a silicone oil, wherein said silicone elastomer crosspolymer is in a volatile fluid. The silicone elastomer crosspolymer can be dimethicone-based or a blend of cylclohexasiloxane and cyclopentasiloxane. The silicone oil can be dimethicone, cyclomethicone or a mixture thereof. The silicone elastomers useful in the wound dressing provided are those that dry quickly, have a soft, silky feel on the skin and add a luxurious texture to dressing when initially applied. The scar dressing provided herein can be occlusive and flexible. The dressings are non-tacky and non-greasy.

Any suitable high molecular weight silicone elastomer may be employed. A silicone elastomer comprises crosslinked silicone polymers. The use of crosslinked silicone polymers eliminates the need for a catalyst or crosslinking agent in the scar dressing formulation. In some embodiments, the preferred molecular weight of the elastomer depends upon the desired viscosity of the scar dressing formulation as well as the desired characteristics of quick drying, conformity, texture, and non-tackiness. Exemplary elastomers include dimethicone crosspolymers as in Dow Corning® 9040 (dimethicone crosspolymer) or KSG-210 (dimethicone/PEG-10/15 crosspolymer and dimethicone) (ShinEtsu Chemical Co. Ltd) as well as Volasil 7525 (a blend of cyclopentasiloxane and cyclohexasiloxane) (Chemisil Silicones Inc.). Typically, the high molecular weight elastomer crosspolymer has a low viscosity of about 50 cSt or less, about 25 cSt or less, or sometimes 5 cSt or less.

The silicone elastomer is in a volatile fluid. In most embodiments, the non-volatile component is less than about 10%, less than about 15%, less than about 20%, or less than about 30% by weight. Volatile fluids include super low viscosity silicone fluids such as cyclomethicone or dimethicone. The silicone elastomer in a volatile fluid represents greater than about 70%, about 80%, greater than about 85%, greater than about 90%, or greater than 95% by weight of the wound dressing formulation.

The silicone oils useful in the wound dressing formulation provided herein have a high nonvolatile content of greater than 70%, greater than 80% or greater than 90%. Exemplary silicone oils include dimethicone, cyclomethicone or a mixture thereof such as Botanisil S-19 (PEG-12 dimethicone). The silicone oil can be in a fluid or powder form. In one embodiment, the silicone oil can be a dimethicone/vinyl dimethicone crosspolymer such as Dow Corning® 9506. The amount of silicone oil in the scar dressing formulation can be from about 0.5% to about 15% of the blend by weight. In some embodiments, the preferred particle size of the elastomer depends upon the desired viscosity of the wound dressing formulation as well as the desired characteristics of quick drying, conformity, texture, and non-tackiness. In general, the particle size range can be from about 500 nm to about 100 μm. In some embodiments, the particle size ranges from about 1 to about 15 μm. The average particle size can be about 500 nm, about 1 μm, about 3 μm, about 5 μm, about 10 μm, about 15 μm, or greater.

The scar dressing formulation may optionally contain one or more additives. Additives include, but are not limited to therapeutic agents, antimicrobials (including antibacterials, antivirals and antifungals), stabilizers, thickeners, pigments, dyes, preservatives and antioxidants. In one embodiment, the scar dressing formulation contains from about 0.001% to about 25-35% by weight of at least one additive. In a particular embodiment, the additive is about 5% or less by weight, about 3% or less by weight, or about 1% or less by weight.

In some embodiments, the additive can increase the smoothness of the scar dressing formulation. Such additives include, but are not limited to glycerin, propylene glycol, butylene glycol, esters, diacyl glycerol esters, and starch.

In some scar dressing formulations, preservatives such as benzyl alcohol are useful. Carriers for therapeutic agents and/or antimicrobials such as water can also be employed.

Stabilizers specifically include amine stabilizers. Suitable thickeners are the swelling agents customarily used for gel formation in galenic pharmacy. Examples of suitable thickeners include natural organic thickeners, such as agar—agar, gelatin, gum arabic, a pectin, etc., modified organic natural compounds, such as carboxymethylcellulose or cellulose ethers, or fully synthetic organic thickeners, such as polyacrylic compounds, vinyl polymers, or polyethers.

The dressings of the presently described invention can be used to deliver a wide variety of compounds and agents. In a preferred embodiment, the compounds are agents that require a hydrophobic carrier or delivery vehicle. In another embodiment, the compounds for delivery are applied using single or multiple dosing regimens, such as daily, or multiple times each day. The presently described formulations are advantageous over other dressing formulations in that the prior art dressing can cause irritation after prolonged exposure. A typical example of such a formulation would be one comprising a retinoid or glycolic acid. Additionally, the presently described dressing formulations are contemplated for use in formulating chemical peels using glycolic acid or tricholoacetic acid.

Pharmacological agents include any bioactive agent including but not limited to antiseptics, antibacterial agents, antifungal agents or other adjuvants employed in burn and wound treatment. Such agents include organic molecules, preferably small organic compounds having a molecular weight of more than 50 and less than about 2,500 daltons; peptides, saccharides, fatty acids, steroids, purines, pyrimidines, derivatives, and structural analogs. Therapeutic agents also include peptide and protein agents, such as antibodies or binding fragments or mimetics thereof, e.g., Fv, F(ab′)₂ and Fab or growth factors, hormones and the like, particularly those that stimulate wound healing and skin growth. Analgesic agents and antibiotics such as phenylbutazone, oxyphenbutazone, indomethacin, naproxen, ibuprofen, acetaminophen, acetylsalicylic acid, penicillins, tetracyclines, and streptomycins are also suitable therapeutic agents useful in the wound dressing provided herein.

Among the agents particularly well suited for use with the presently described invention this include those agents that are unstable in water based systems including nitroglycerin, retinoic acid and its derivatives, vitamin D and its derivatives, acetyl salicylic acid, vitamin C, and some antibiotics, including tetracyclines, mupirocin, and cephalosporins.

Another distinct advantage of this drug delivery vehicle is its use with agents that are unstable in the presence of surfactants or where surfactants can cause damage to the drug over time, e.g. proteins, peptides and linked amino acids. The presently disclosed invention does not require the use of potentially harsh or denaturing surfactants, like sodium laural sulfate (SLS), sodium laureth sulfate (SLES), and ammonium laural sulfate (ALS), cocamidopropyl betaine, which are known to cause considerable irritation to those sensitive to them, it is ideal for formulation of drug products for patients with sensitive skin.

In formulations used to treat acne for example sensitive skin is very common and incorporating an anti-acne drug like benzoyl peroxide. One of the afflictions of psoriasis, in addition to the disfigurement of the disease itself, is tremendous skin sensitivity. Topically medications like corticosteroids, or calcipotriene are ideally incorporated into this non-irritating, easy to apply drug delivery vehicle. The term “corticosteroid” includes glucocorticoids as well as mineralocorticoids. Examples of corticosteroids include aclometasone dipropionate, amcinonide, betamethasone dipropionate, betamethasone sodium phosphate, betamethasone valerate, budesonide, clobetasol-17-propionate, clobetasone-17-butyrate, cortisone acetate, desonide, dexamethasone, dexamethasone sodium phosphate, fluocinolone acetonide, fluocinonide, fluocortolone, fluocortolone caproate, fluocortolone pivalate, fluprednidene acetate., halcinonide, hydrocortisone, hydrocortisone acetate, hydrocortisone-17-butyrate, hydrocortisone-17-valerate, methylprednisolone, mometasone, prednicarbate, prednisolone, prednisone, tixocortol pivalate, triamcinolone acetonide, and triamcinolone alcohol.

Drug categories in which this drug delivery vehicle can be use in are antihistamines, anti-infectives, anti-inflammatory agents including corticosteroids, anti-psoriatic agents, arnica, chloracetic acid, podofilox, and podophyllium resing, methionine, aluminum chloride hexahydrate, collagenase, hyaluronidase, ketatolytic agents, local anesthetics, minoxidil, psoralens, pigment agents including hydroquinone and monobenzone, 5-FU, retinoids, scabicides and pediculicides, wound healing agents, urea, dexpanthenol, Vitamin E, and sunscreens.

In a particular embodiment, the scar dressing formulation provided herein includes liposomes or liposomal compositions. Any suitable liposome or liposome composition may be employed. In some embodiments, the liposomes contain one or more therapeutic agents suitable for a wound dressing. Exemplary liposomes include those in U.S. Pat. Nos. 6,958,160 and 7,150,883. In one embodiment, the liposome comprises one or more lipids that is a diacylglycerol-PEG, particularly dioleolylglycerol-PEG-12.

The formulation is useful at any stage during scar evolution. The formulation can be applied to a wound that has completed the initial re-epithelization process or is a closed wound. The formulation is also useful to treat scars during the contraction, maturation or remodeling stages of wound healing. Thus, the scar can be less than about 1 week old, about 2 weeks old, about 1 month old, about 3 months old, or greater. Scars resulting from any type of wound may be treated in accordance with the present invention. Such scars include but are not limited to those resulting from or related to cuts, abrasions, traumatic skin injury, burns, and surgical wounds (such as those resulting from the use of a scalpel or a laser). Such scars can be atrophic, hypertrophic, keloid or contracture. The scar dressing formulation provided herein is particularly adapted for the treatment and/or prevention of hypertrophic scars of wounds following burn injuries.

In some embodiments, the scar dressing formulation is applied to the desired site while in a substantially flowable state. Once the scar dressing formulation is completely blended, it remains flowable and thus applicable to wound surfaces for up to 15 minutes. The flowable or substantially flowable state permits the wound dressing formulation to be custom fit to any contoured or shaped surface. Thus, the formulation is applied to the scar and can be worked with for about 2 minutes to about 15 minutes to cover the scar as necessary. After application, the scar dressing formulation is smoothed to a desired thickness and is substantially tack-free after mixing.

The scar dressing formulation typically forms a membrane having a thickness from about 0.1 mm to about 5 mm upon curing. The membrane can be continuous or substantially continuous over the surface of the scar. The continuous nature of the membrane allows the scar dressing to retain moisture in the scar as well as act as a bacterial barrier. The scar dressing is free or at least substantially free of air bubbles.

The scar dressing formulation can be transparent or substantially transparent. Transparency permits visual observation and monitoring of the scar as it continues to heal and improves the cosmetic appearance of the dressing (e.g., renders it less conspicuous).

The scar dressing formulation can remains on the scar for any time sufficient to permit healing of and/or resolution of the scar. In one embodiment, the scar dressing formulation forming a membrane remains on the wound at least about 1 day, at least about 2 days, at least about 4 days, at least about 6 days, or at least about 7 days to about 10 days.

After the scar dressing formulation has been on a scar for a time sufficient to promote and/or substantially complete healing and scar formation, the scar dressing can removed by gently wiping it from the scar. The healed wound is characterized by decreased redness, moistness, and minimal scarring.

Further provided herein is a kit comprising the components of the formulation as disclosed herein and optionally instructions for use.

A number of exemplary formulations are provided below.

Retinoic Acid

| | |
|---|---|
| Dow corning 9040 | 83.5% |
| Cyclomethicone | 13.50% |
| Propylene glycol | 1.45% |
| PEG-12 glyceryl dimyristate | 0.05% |
| Retinoic Acid | 0.05% |
| Benzyl Alcohol | 1.00% |

Glycolic Acid Peel

| | |
|---|---|
| Dow corning 9040 | 60.5% |
| Cyclomethicone | 15.00% |
| Glycerin | 1.00% |
| PEG-12 glyceryl dimyristate | 1.5% |
| Glycolic acid | 20.00% |
| Benzyl Alcohol | 2.00% |

Vitmain C

| | |
|---|---|
| Volasil 725 | 80.5% |
| Dow coming Powder 9506 | 7.0% |
| Glycerin | 1.00% |
| PEG-12 glyceryl dimyristate | 1.00% |
| Vitamin C | 9.50% |
| Benzyl Alcohol | 1.00% |

Mupirocin

| | |
|---|---|
| Volasil 725 | 79.5% |
| Cyclomethicone | 15.0% |
| Glycerin | 1.00% |
| PEG-12 glyceryl dimyristate | 1.00% |
| Mupirocin | 2.0% |
| Benzyl Alcohol | 1.50% |

Somatostatin (Octreotide)

| | |
|---|---|
| Dow corning 9040 | 85.5% |
| Cyclomethicone | 10.00% |
| Glycerin | 0.95% |
| PEG-12 glyceryl dimyristate | 1.5% |
| Octreotide acetate | 0.05% |
| Benzyl Alcohol | 2.00% |

Clobetasol Proprionate

| | |
|---|---|
| Dow corning 9040 | 85.5% |
| Cyclomethicone | 10.00% |
| Propylene glycol | 0.95% |
| Ethanol | 1.5% |
| Clobetasol proprionate | 0.05% |
| Benzyl Alcohol | 2.00% |

Diphenhydramine

| | |
|---|---|
| Volasil 725 | 80.5% |
| Dow coming Powder 9506 | 13.5% |
| Glycerin | 1.00% |
| PEG-12 glyceryl dimyristate | 1.00% |
| Diphenhydramine HCl | 2.00% |
| Benzyl Alcohol | 2.00% |

The following examples are offered to illustrate but not to limit the invention.

### EXAMPLE 1

### Scar Dressing Formulation Using Dimethicone Crosspolymers

KSG-210 is a dimethicone and dimethicone/PEG-10/15 crosspolymer that swells in silicone fluid. Minute cross-linked particles orient at the interface with fluid and to form a network. Botanisil S-19 is a silicone oil that is PEG-12 dimethicone.

GDM-12 is a specific type of self-forming, thermodynamically stable liposomes suitable for delivery of therapeutic agents (see U.S. Pat. No. 6,958,160). More specifically, GDM-12 is a mixture of liposomes formed from glycerol dimyristate (“GDM”) lipids where the head group of the lipid includes a polyethylene glycol (“PEG”) molecule with 12 C₂H₄O subunits in the PEG chain.

| | | |
|---|---|---|
| KSG-210 | 96.7500% | 96.7500 |
| Botanisil S-19 | 0.5000% | 0.5000 |
| GDM-12 | 0.5000% | 0.5000 |
| Benzyl Alcohol | 0.7500% | 0.7500 |
| Purified Water | 1.5000% | 1.5000 |
| Total: | 100.0000% | 100.0000 |

### EXAMPLE #2

### Scar Dressing Formulation Using Dimethicone Crosspolymers

Dow Corning® 9040 Silicone Elastomer Blend is a mixture of a high molecular weight silicone elastomer (i.e., dimethicone crosspolymer) in cyclomethicone (<1 wt %). It is a volatile diluent that is a silicon fluid. It has a viscosity range of 250,000-580,000 cp and a typical nonvolatile content of 12.0 wt % to 12.75 wt %. Cyclomethicone is a silicone oil.

| | | | |
|---|---|---|---|
| 1 | Dow Corning 9040 | 83.5000% | 83.5000 |
| 2 | Cyclomethicone | 15.0000% | 15.0000 |
| 3 | Glycerin | 1.0000% | 1.0000 |
| 4 | GDM-12 | 0.5000% | 0.5000 |
| | Total: | 100.0000% | 100.0000 |

### EXAMPLE #3

### Scar Dressing Formulation Using Dimethicone Crosspolymers

Volasil 7525 is a low viscosity mixture of the elastomers cyclohexasiloxane and cyclopentasiloxane. Dow Corning® 9506 powder is a silicone oil. More particularly, Dow Corning® 9506 is a dimethicone/vinyl dimethicone crosspolymer with a non-volatile content of 98% (minimum). It is a white free-flowing powder that provides dry smoothness and a powdery-light non-greasy skin feel. It also reduces tackiness.

| | | | |
|---|---|---|---|
| 1 | Volasil 7525 | 85.5000% | 85.5000 |
| 2 | Dow Corning ® 9506 powder | 13.0000% | 13.0000 |
| 3 | Glycerin | 1.0000% | 1.0000 |
| 4 | GDM-12 | 0.5000% | 0.5000 |
| | Total: | 100.0000% | 100.0000 |

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

1. A scar dressing formulation comprising
a high molecular weight, low viscosity silicone crosspolymer in a volatile fluid; and
a silicone fluid/oil.
